(19) United States
(12) Patent Application Publication
(10) Pub. No.: US 2018/0028589 A1
(43) Pub. Date: Feb. 1, 2018

(54) Pain Relief Composition

(71) Applicant: Carolyn Wallace, Leesburg, GA (US)

(72) Inventor: Carolyn Wallace, Leesburg, GA (US)

(21) Appl. No.: 15/652,791

(22) Filed: Jul. 18, 2017

Related U.S. Application Data
(60) Provisional application No. 62/368,547, filed on Jul. 29, 2016.

(51) Int. Cl. A61K 36/899; A61K 36/53; A61K 36/889

(52) U.S. Cl. CPC A61K 36/899; A61K 36/889; A61K 36/53

(57) ABSTRACT

A pain relief composition including natural oils is provided. The composition includes an ointment formed by a mixture of essential oils including lavender oil, lemongrass oil, and rosemary oil along with solid coconut oil. The essential oils are naturally occurring ingredients extracted from various plants that can be used on the body, without causing any side effects. The oils contain numerous therapeutic qualities including analgesic, antibacterial, anti-inflammatory, cicatrisant, diuretic, sedative and hypotensive properties. This combination of coconut oil along with the essential oils forms an ointment which provides a user with temporary relief from pain caused by physical and mental conditions, such as arthritis, migraines, anxiety, gout, and flatulence.

### CROSS REFERENCE TO RELATED APPLICATIONS

This application claims the benefit of U.S. Provisional Application No. 62/368,547 filed on Jul. 29, 2016. The above identified patent application is herein incorporated by reference in its entirety to provide continuity of disclosure.

### FIELD OF THE INVENTION

The present invention relates to chemical compositions. More specifically, the present invention provides a pain relief composition including coconut oil and essential oils for use as an ointment that provides pain relief and other therapeutic properties.

### BACKGROUND OF THE INVENTION

Many individuals suffer from various physical and/or mental conditions on a daily basis, which in turn affects their quality of life. This may mean they are unable to function at full capacity, need daily assistance, and lose some of their independence. Some methods of relieving physical and mental pain include taking prescription medications. However, this medication is often highly addictive, and often has many undesirable side effects that may be worse than the pain itself. Other methods include applying heat or ice to the area experiencing pain. However, heat or ice alone is insufficient to relieve chronic pain or particularly intense pain. There are also many substances presently existing which can be applied topically to an area experiencing pain. However, these substances must be applied in copious amounts. Many of these substances often contain chemicals which can be harmful to the skin when applied frequently. It is therefore desirable to provide a pain relief composition consisting of essential oils that can be used to alleviate pain throughout the body, as well as reduce swelling and scarring.

Essential oils possess numerous properties which make them useful for treating many of our most common health and beauty troubles. The oils are extracted from flowers, stems, leaves, roots and barks of aromatic plants. The most common method of extracting the oils from the plants is by distillation. Essential oils are complex mixtures of various organic molecules, such as alcohols, esters, aldehydes, phenols, and terpenes. Many of the presently existing ointments contain synthetic imitations of essential oils, however, while the synthetics have a similar aromatic quality, they do not possess the same therapeutic properties.

Coconut oil is predominantly composed of saturated fatty acids along with a few medium chain fatty acids. The saturated fats possess antioxidants and anti-aging properties by countering the effects of aging, whitening of hair, and sagging of the skin. Additionally, upon reaction with certain enzymes within the body, the acids can convert themselves into antimicrobial agents such as monocaprin and monolaurin. These chemical derivatives protect the body both internally and externally from conditions such as rashes, ringworm, and dermatitis. Coconut oil also possesses a significant amount of moisture retaining capacity, making the oil ideal for skin care use. Another primary constituent of coconut oil is Vitamin-E, which helps keep hair and skin healthy and keeps your organic system functioning smoothly. The oil is also effective in aiding the absorption of fat-soluble vitamins and glucose, and improves circulation and contains saturated fatty acids which help protect against certain types of cancers. Therefore, there is a need for an ointment consisting of coconut oil along with essential oils possessing medicinal qualities.

The present invention is an ointment which includes a mixture of 100% natural coconut oil and essential oils. The ointment contains analgesic properties designed to numb pain, cicatrisant properties for skin regeneration and to heal scars, diuretic properties to increase urination, hypotensive properties to lower blood pressure, and sedative properties to calm and tranquilize. The ointment can also be used for anti-inflammatory, antirheumatic, and antibacterial purposes. The combination of these properties creates an ointment which improves several physical and mental conditions. These conditions include aching muscles and joints, arthritis, rheumatism, depression, insomnia, anxiety and emotional stress, stretch marks, sprains and strains, migraines, tension, poor circulation, neuralgia, gout, eczema and psoriasis, hypertension, vertigo, and flatulence.

In light of devices disclosed in the prior art, it is submitted that the present invention substantially diverges in design elements from the prior art and consequently is clear that there is a need in the art for an improvement to existing pain relief devices and compositions. In this regard, the instant invention substantially fulfills these needs.

### SUMMARY OF THE INVENTION

In view of the foregoing disadvantages inherent in the known types of pain relief compositions now present in the prior art, the present invention provides a pain relief oil composition wherein the same can be utilized for providing convenience for the user when in need of an ointment having natural components that alleviates pain caused by physical and mental diseases. The pain relief oil composition comprises solid coconut oil, lavender oil, lemongrass oil, and rosemary oil. The combination of oils allows individuals to treat any type of body pain, such as pain and discomfort caused by arthritis, inflammation, injuries, anxiety, migraines, gout, depression, neuralgia and a variety of other ailments.

One object of the present invention is to provide an oil composition for relieving pain caused by physical and mental conditions and providing other therapeutic benefits.

Another object of the present invention is to provide a pain relief oil composition having a pleasant fragrance.

A further object of the present invention is to provide a pain relief oil composition that includes only natural ingredients.

Other objects, features and advantages of the of the present invention will become apparent from the following detailed description.

### BRIEF DESCRIPTIONS OF THE DRAWINGS

Although the characteristic features of this invention will be particularly pointed out in the claims, the invention itself and manner in which it may be made and used may be better understood after a review of the following detailed description.

### DETAILED DESCRIPTION OF THE INVENTION

For the purposes of presenting a brief and clear description of the present invention, the preferred embodiment will be discussed as used for preparing an ointment for relieving pain and other therapeutic purposes. The following detailed description is intended for representative purposes only and should not be considered limiting in any respect.

The present invention provides a pain relief composition configured to provide pain relief and other therapeutic benefits, such as reduction of inflammation and scarring, lowering of high blood pressure, and destruction of harmful bacteria. The composition includes a mixture of oils including coconut oil, lavender oil, lemongrass oil, and rosemary oil. The combination of coconut oil and essential oils provides a fragrance with aroma therapy benefits along with other therapeutic properties to the pain relief ointment.

In one embodiment, the pain relief composition comprises 90.0% to 98.5% by weight of solid coconut oil, 0.4% to 2.5% by weight of lavender oil, 0.4% to 2.5% by weight of lemongrass oil, and 0.1% to 1.5% by weight of rosemary oil. This specific combination of ingredients is configured to provide the maximum amount of therapeutic properties.

In order to compose the pain relief composition, the measured amount of solid coconut oil is mixed until it is a smooth texture. The measured amounts of lavender oil, lemongrass oil, and rosemary oil are stirred into the smooth coconut oil. The composition is then packaged into a suitable jar or container. A safety seal liner is placed on top of the jar prior to screwing on a lid.

In order to prepare the pain relief composition, an individual may apply the ointment to the affected areas of the body. The composition of oils allows the individual to obtain temporary relief from aches and pains associated with arthritis, sprains, rheumatism, gout, insomnia, hypertension, migraines, and other forms of pain produced by both physical and mental diseases. The composition also reduces anxiety, vertigo, inflammation, swelling, and scarring.

It is therefore submitted that the instant invention has been shown and described in what is considered the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to falling within the scope of the invention.

I claim:

1. A pain relief oil composition consisting essentially of:
coconut oil;
lavender oil;
lemongrass oil;
rosemary oil.

2. The pain relief oil composition of claim 1, wherein the amount of the coconut oil in the composition is within a range of 90.0% to 98.5% by weight.

3. The pain relief oil composition of claim 2, wherein the amount of the lavender oil in the composition is within a range of 0.4% to 2.5% by weight.

4. The pain relief oil composition of claim 3, wherein the amount of the lemongrass oil in the composition is within a range of 0.4% to 2.5% by weight.

5. The pain relief oil composition of claim 4, wherein the amount of the rosemary oil in the composition is within a range of 0.1 to 1.5% by weight.

6. A pain relief oil composition consisting essentially of:
90.0 to 98.5 percent by weight of coconut oil;
0.4 to 2.5 percent by weight of lavender oil;
0.4 to 2.5 percent by weight of lemongrass oil;
0.1 to 1.5 percent by weight of rosemary oil.
